(19) United States
(12) Patent Application Publication
(10) Pub. No.: US 2020/0330576 A1
(43) Pub. Date: Oct. 22, 2020

(54) ALDH1 ANTIGEN-PULSED DENDRITIC CELLS

(71) Applicant: The Regents of the University of Michigan, Ann Arbor, MI (US)

(72) Inventor: Qiao Li, Ann Arbor, MI (US)

(21) Appl. No.: 16/957,336

(22) PCT Filed: Jan. 3, 2019
(86) PCT No.: PCT/US19/12191

§ 371 Date: Jun. 23, 2020

Related U.S. Application Data
(60) Provisional application No. 62/614,591, filed on Jan. 8, 2018.

(51) Int. Cl. A61K 39/00; C12N 5/0784; A61P 35/00

(52) U.S. Cl. CPC A61K 39/001154; A61K 45/06; A61P 35/00; C12N 5/0639

(57) ABSTRACT

The present invention relates to compositions, systems, kits, and methods for generating and using ALDH1 anti-gen-pulsed dendritic cells (DCs). In certain embodiments, initial DCs are pulsed in vitro with a composition comprising ALDH1A1 and/or ALDH1A3 immunogenic peptide(s) to generate ALDH1 antigen-pulsed DCs, wherein the composition is free of tumor cells, cell lysates, and full-length ALDH1 proteins. In some embodiments, the ALDH1 antigen-pulsed DCs are administered to a subject in order to at least partially treat cancer (e.g., to kill at least some cancer stem cells in the subject).

The present application claims priority to Provisional Application Ser. No. 62/614,591, filed Jan. 8, 2018, which is herein incorporated by reference in its entirety.

### FIELD OF THE INVENTION

The present invention relates to compositions, systems, kits, and methods for generating and using ALDH1 antigen-pulsed dendritic cells (DCs). In certain embodiments, initial DCs are pulsed in vitro with a composition comprising ALDH1A1 and/or ALDH1A3 immunogenic peptides to generate ALDH1 antigen-pulsed DCs, wherein the composition is free of tumor cells, cell lysates, and full-length ALDH1 proteins. In some embodiments, the ALDH1 antigen-pulsed DCs are administered to a subject in order to at least partially treat cancer (e.g., to kill at least some ALDH^{high} cancer stem cells in the subject).

### BACKGROUND OF THE INVENTION

Clinical trials to treat patients with cancer using adoptively transferred T cells or dendritic cells have shown therapeutic efficacy for patients with advanced diseases. However, the clinical responses to such immunotherapeutic approaches have been confined to a limited percentage of treated patients. Generally, bulk tumor masses with heterogeneous populations of cancer cells have been used as a source of antigen either to generate effector T cells or to prime DC vaccines. Human tumors are composed of heterogeneous tumor cell clones that differ with respect to proliferation, differentiation, and ability to initiate daughter tumors. The inability to target cancer stem cells (CSC) with current immune approaches may be a significant factor for treatment failures.

The identification of human CSCs presents a new paradigm for the development of cancer treatments. These stem cells have been shown to be relatively resistant to conventional chemotherapeutic regimens and radiation and are postulated to be the cells responsible for the relapse and progression of cancers after such therapies. In an analogous fashion, the CSC phenomenon may adversely affect the development of effective immunotherapies for cancer. These therapies have involved targeting cells that express differentiated tumor antigens. However, such antigens may be selectively expressed on differentiated tumor cells. CSCs that do not express these antigens may thus escape these immunologic interventions.

### SUMMARY OF THE INVENTION

The present invention provides compositions, systems, kits, and methods for generating and using ALDH1 antigen-pulsed dendritic cells (DCs). In certain embodiments, initial DCs are pulsed in vitro with a composition comprising ALDH1A1 and/or ALDH1A3 immunogenic peptides to generate ALDH1 antigen-pulsed DCs, wherein the composition is free of tumor cells, cell lysates, and full-length ALDH1 proteins. In some embodiments, the ALDH1 antigen-pulsed DCs are administered to a subject in order to at least partially treat cancer (e.g., to kill at least some ALDH^{high} cancer stem cells in the subject).

In some embodiments, provided herein are methods of generating antigen-pulsed dendritic cells comprising: contacting (e.g., loading) initial dendritic cells (DCs) in vitro with a composition comprising ALDH1A1 and/or ALDH1A3 (e.g., human ALDH1A1 and/or ALDH1A3) immunogenic peptides that are 8 to 100 or 8 to 250 amino acids in length, wherein the composition is free (e.g., detectably free) of: i) full-length ALDH1A1 and ALDH1A3 proteins, and ii) tumor cells and cell-lysates or tumor cell-lysates. In particular embodiments, the methods further comprise, prior to the contacting, i) collecting the initial DCs from a subject (e.g., human subject) and, ii) culturing the initial DCs (e.g., with IL-4 and/or GM-CSF). In certain embodiments, the collecting comprises isolating the initial DCs from blood (e.g., human) or bone marrow from the subject (e.g., an animal).

In particular embodiments, provided herein are methods of treating cancer in a subject comprising: administering ALDH1A antigen-pulsed dendritic cells (DCs) to a subject having cancer cells such that at least some of the cancer cells (e.g., ALDH^{high} cancer cells) are killed (e.g., any tumor is reduced in size, or the total population size of cancer cells is reduced in number, or the tumor relapse is reduced, or metastasis is reduced with increased host survival), wherein the antigen-pulsed DCs are initial DCs that have been pulsed in vitro with a composition comprising human ALDH1A1 and/or ALDH1A3 immunogenic peptides that are 8 to 100, or 8 to 250, amino acids in length, wherein the composition is free of: i) full-length ALDH1A1 and ALDH1A3 proteins, and ii) tumor cells and cell-lysates. In particular embodiments, the initial DCs are from the subject to be treated. In other embodiments, the subject has previously had a solid tumor removed (e.g., surgical removal of one or more visible tumors). In certain embodiments, the administering to the subject increases the length of survival of the subject compared to the length of survival without the administering. In other embodiments, the method further comprises: administering an immune checkpoint inhibitor to the subject (e.g., an inhibitor of PD-1 or PD-L1). In certain embodiments, the subject is a human.

In other embodiments, provided herein are compositions comprising: dendritic cells (DCs), and human ALDH1A1 and/or ALDH1A3 immunogenic peptides that are 8 to 100, or 8-250, amino acids in length, wherein the composition is free of: i) full-length ALDH1A1 and ALDH1A3 proteins, and ii) tumor cells and cell-lysates.

In some embodiments, provided herein are compositions comprising: antigen-pulsed DCs which are initial DCs that have been pulsed in vitro with a pulsing composition comprising human ALDH1A1 and/or ALDH1A3 immunogenic peptides that are 8 to 100, or 8 to 250, amino acids in length, wherein the pulsing composition is free of: i) full-length ALDH1A1 and ALDH1A3 proteins, and ii) tumor cells and cell-lysates. In certain embodiments, the compositions further comprise a physiologically tolerable buffer.

In other embodiments, provided herein are systems and kits comprising: a) dendritic cells (DCs), and b) a composition comprising human ALDH1A1 and/or ALDH1A3 immunogenic peptides that are 8 to 100 amino acids in length, wherein the composition is free of: i) full-length ALDH1A1 and ALDH1A3 proteins, and ii) tumor cells and cell-lysates. In certain embodiments, the compositions further comprise a physiologically tolerable buffer. In other embodiments, the systems and kits further comprise: c) culture medium (e.g., comprising IL-4 and/or GM-CSF).

In certain embodiments, the initial DCs comprise immature DCs. In further embodiments, the human ALDH1A1 and/or ALDH1A3 immunogenic peptides are between 8 and 50 amino acids in length (e.g., 8 . . . 15 . . . 37 . . . or 50 amino acids in length). In certain embodiments, the human ALDH1A1 and/or ALDH1A3 immunogenic peptides are a portion of human ALDH1A1, accession no. NM_000689; SEQ ID NO:61, or a portion of human ALDH1A3, accession No. NM_000693, SEQ ID NO:62). In some embodiments, the human ALDH1A1 and/or ALDH1A3 immunogenic peptides are between 8 and 23 amino acids in length (e.g., 8 . . . 10 . . . 12 . . . 15 . . . 19 . . . 21 . . . and 23 amino acids in length). In some embodiments, the human ALDH1A1 and/or ALDH1A3 immunogenic peptides are between 8 and 10 amino acids in length (e.g., exactly 8, 9, or 10 amino acids in length).

In some embodiments, the composition is further free of ALDH1A1 and ALDH1A3 peptides larger than 250 or larger than 100 amino acids in length. In certain embodiments, the composition is further free of ALDH1A1 and ALDH1A3 peptides larger than 35 amino acids in length. In other embodiments, the composition is further free of ALDH1A1 and ALDH1A3 peptides larger than 10 amino acids in length. In particular embodiments, the ALDH1A1 and/or ALDH1A3 immunogenic peptides comprise or consist of an amino acid sequence shown in SEQ ID NOS:1-60. In certain embodiments, the ALDH1A1 and/or ALDH1A3 immunogenic peptides comprise or consist of the amino acid sequences shown in SEQ ID NOS:1 and/or 6. In further embodiments, the ALDH1A1 and/or ALDH1A3 immunogenic peptides, collectively, are present in the composition at a concentration of at least 50 μg/ml (e.g. at least 50 . . . 100 . . . 150 . . . 200 . . . 250 . . . 300 . . . 350 . . . 400 . . . 450 . . . 500 . . . 550 . . . 650 . . . 850 . . . 1000 μg/ml or more).

In certain embodiments, the subject that is administered antigen-pulsed DCs has a cancer selected from the group consisting of: melanoma, breast cancer, prostate cancer, pancreatic cancer, lung cancer, liver cancer, brain cancer, skin cancer, squamous cell carcinoma, and colon cancer. In further embodiments, the methods further comprise treating the subject with a chemotherapeutic agent. In other embodiments, the methods further comprise treating the subject with radiation treatment. In particular embodiments, the cancer cells are cancer stem cells.

In further embodiments, the subject has a cancer selected from the group consisting of: melanoma, breast cancer, prostate cancer, pancreatic cancer, lung cancer, liver cancer, brain cancer, head and neck squamous cell carcinoma, skin cancer, and colon cancer. In other embodiments, the methods further comprise further treating the subject with an immunological agent (e.g., anti-PD-1 or anti-PD-L1 antibody). In other embodiments, the methods further comprise further treating with chemotherapeutic agent (e.g., small molecule). In other embodiments, the methods further comprise further treating with radiation therapy (e.g., external beam radiation therapy). In certain embodiments, the radiation therapy comprises internal radiation therapy. In other embodiments, the methods further comprise further treating the subject with prior surgical removal of the tumor.

### DESCRIPTION OF THE DRAWINGS

FIG. 1 shows the procedure from Example 1 for generating ALHD1A1 and/or ALDH1A3 peptide(s) exposed DCs to activate T-cells.

FIG. 2 shows the cytotoxicity of CD3+ T cells stimulated in vitro with ALDH1A1 and/or ALDH1A3 peptide(s)-DCs against ALDHhigh CSC vs. ALDH^{low} non-CSC targets.

FIG. 3 shows the protocol from Example 2 for preventing tumor growth in vivo with ALHD1A1 and/or ALDH1A3 peptide(s)-DC vaccine.

FIG. 4 shows how the ALDH1A1 or ALDH1A3 peptide-DC vaccine demonstrated significant suppressive effect on D5 tumor growth.

FIG. 5 shows how the combined ALDH1A1 and 1A3 peptides-DC vaccine demonstrated increased suppressive effect on D5 tumor growth.

FIG. 6 shows how the ALHD1A1 and/or ALDH1A3 peptide(s) -DC vaccine demonstrated increased suppressive effect on D5 tumor.

FIG. 7 shows how the CD3+T cells isolated from the TILs of D5-bearing mice treated with ALDH 1A1 or 1A3 peptide-DC vaccine demonstrated significantly elevated killing of D5 ALDH^{high} CSCs.

FIG. 8 shows the cytotoxicity of spleen T cells isolated from D5-bearing mice treated with ALDH 1A1 and/or 1A3 peptides-DC vaccine, as they demonstrated significant killing effect on D5 ALDH^{high} CSCs.

FIG. 9, second row, shows flow cytometry scatter plots of intracellular staining of IFN-γ secreted by ALDH 1A1 and/or 1A3 peptide(s)-DC vaccine-primed spleen T cells in response to ALDH^{high} D5 CSCs. The first row shows flow cytometry scatter plots of isotype control for the anti-IFN-γ monoclonal antibody.

FIG. 10, second row, shows flow cytometry scatter plots of intracellular staining of IFN-γ secreted by ALDH 1A1 and/or 1A3 peptide(s)-DC vaccine-primed spleen T cells in response to ALDH^{low} D5 non-CSCs. The first row shows flow cytometry scatter plots of isotype control for the anti-IFN-γ monoclonal antibody.

FIG. 11 shows the amino acid sequence of full-length human ALDH1A1 (NM_000689), which is SEQ ID NO:61. A box is shown around ALDH1A1 peptide SEQ ID NO:1.

FIG. 12 shows the amino acid sequence of full-length human ALDH1A3 (NM_000693), which is SEQ ID NO:62. A box is shown around ALDH1A3 peptide SEQ ID NO:6.

### DEFINITIONS

As used herein, the term “subject” refers to any animal (e.g., a mammal), including, but not limited to, humans, non-human primates, rodents, and the like (e.g., which is to be the recipient of a particular treatment, or from whom cancer stem cells are harvested). Typically, the terms “subject” and “patient” are used interchangeably, unless indicated otherwise herein.

As used herein, the term “subject is suspected of having cancer” refers to a subject that presents one or more signs or symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis (e.g., a CT scan showing a mass) but for whom a confirmatory test (e.g., biopsy and/or histology) has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, blood test, and the diagnostic methods of the present invention. A “preliminary diagnosis” is one based only on visual (e.g., CT scan or the presence of a lump) and antigen tests.

As used herein, the term “effective amount” refers to the amount of a composition or treatment sufficient to effect beneficial or desired results. An effective amount can be administered in one or more administrations, applications or dosages and is not intended to be limited to a particular formulation or administration route. In certain embodiments, a subject is administered an effective amount of ALDH1 peptide-DCs.

As used herein, the term “administration” refers to the act of giving a ALDH1 peptide-DC vaccine, drug, prodrug, or other agent, or therapeutic treatment to a subject. Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Co-administration” refers to administration of more than one chemical agent or therapeutic treatment (e.g., radiation therapy) or surgery or immune check point (e.g., PD-1/PD-L1) inhibitor to a physiological system (e.g., a subject or in vivo, in vitro, or ex vivo cells, tissues, and organs). “Co-administration” of the respective chemical agents and therapeutic treatments (e.g., radiation therapy) or surgery or immune check point inhibitor (e.g., PD-1/PD-L1) may be concurrent, or in any temporal order or physical combination.

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds.

### DETAILED DESCRIPTION OF THE INVENTION

The present invention relates to compositions, systems, kits, and methods for generating and using ALDH1 antigen-pulsed dendritic cells (DCs). In certain embodiments, initial DCs are pulsed in vitro with a composition comprising ALDH1A1 and/or ALDH1A3 immunogenic peptides to generate ALDH1 antigen-pulsed DCs, wherein the composition is free of tumor cells, cell lysates, and full-length ALDH1 proteins. In some embodiments, the ALDH1 antigen-pulsed DCs are administered to a subject in order to at least partially treat cancer (e.g., to kill at least some ALDH^{high} cancer stem cells in the subject).

In certain embodiments, an ALDH1A1 or ALDH1A3 peptide (e.g., 8-50 amino acids in length) is employed that comprises or consists of at least one of the amino acid sequences shown in SEQ ID NOS:1-60, which are shown in Table 1 below.

TABLE 1
Peptides from ALDH1A1 and ALDH1A3

| | Human | | SEQ |
|---|---|---|---|
| Sequence | Protein | Length | ID NO: |
| LLYKLADLI | ALDH1A1 | 9 | 1 |
| LLYKLADL | ALDH1A1 | 8 | 2 |
| LYKLADLI | ALDH1A1 | 8 | 3 |
| RLLYKLADLI | ALDH1A1 | 10 | 4 |
| LLYKLADLIM | ALDH1A1 | 10 | 5 |
| LLHQLADLV | ALDH1A3 | 9 | 6 |
| LLHQLADL | ALDH1A3 | 8 | 7 |
| LHQLADLV | ALDH1A3 | 8 | 8 |
| RLLHQLADLV | ALDH1A3 | 10 | 9 |
| LLHQLADLVE | ALDH1A3 | 10 | 10 |
| ASERGRLLY | ALDH1A1 | 9 | 11 |
| SERGRLLY | ALDH1A1 | 8 | 12 |
| ASERGRLL | ALDH1A1 | 8 | 13 |
| DASERGRLLY | ALDH1A1 | 10 | 14 |
| ASERGRLLYK | ALDH1A1 | 10 | 15 |
| RLLYKLADL | ALDH1A1 | 9 | 16 |
| LLYKLADL | ALDH1A1 | 8 | 17 |
| RLLYKLAD | ALDH1A1 | 8 | 18 |
| GRLLYKLADL | ALDH1A1 | 10 | 19 |
| RLLYKLADLI | ALDH1A1 | 10 | 20 |
| ASERGRLLY | ALDH1A1 | 9 | 21 |
| SERGRLLY | ALDH1A1 | 8 | 22 |
| ASERGRLL | ALDH1A1 | 8 | 23 |
| DASERGRLLY | ALDH1A1 | 10 | 24 |
| ASERGRLLYK | ALDH1A1 | 10 | 25 |
| KLIKEAAGK | ALDH1A1 | 9 | 26 |
| LIKEAAGK | ALDH1A1 | 8 | 27 |
| KLIKEAAG | ALDH1A1 | 8 | 28 |
| GKLIKEAAGK | ALDH1A1 | 10 | 29 |
| KLIKEAAGKS | ALDH1A1 | 10 | 30 |
| GLSAGVFTK | ALDH1A1 | 9 | 31 |
| LSAGVFTK | ALDH1A1 | 8 | 32 |
| GLSAGVFT | ALDH1A1 | 8 | 33 |
| YGLSAGVFTK | ALDH1A1 | 10 | 34 |
| GLSAGVFTKD | ALDH1A1 | 10 | 35 |
| ALYLGSLIK | ALDH1A3 | 9 | 36 |
| LYLGSLIK | ALDH1A3 | 8 | 37 |
| ALYLGSLI | ALDH1A3 | 8 | 38 |
| TALYLGSLIK | ALDH1A3 | 10 | 39 |
| ALYLGSLIKE | ALDH1A3 | 10 | 40 |
| ALAEYTEVK | ALDH1A3 | 9 | 41 |
| LAEYTEVK | ALDH1A3 | 8 | 42 |
| ALAEYTEV | ALDH1A3 | 8 | 43 |
| YALAEYTEVK | ALDH1A3 | 10 | 44 |
| ALAEYTEVKT | ALDH1A3 | 10 | 45 |
| RLLHQLADL | ALDH1A3 | 9 | 46 |
| LLHQLADL | ALDH1A3 | 8 | 47 |
| RLLHQLAD | ALDH1A3 | 8 | 48 |
| GRLLHQLADL | ALDH1A3 | 10 | 49 |
| RLLHQLADLV | ALDH1A3 | 10 | 50 |
| ALPRPIRNL | ALDH1A3 | 9 | 51 |
| LPRPIRNL | ALDH1A3 | 8 | 52 |
| ALPRPIRN | ALDH1A3 | 8 | 53 |
| PALPRPIRNL | ALDH1A3 | 10 | 54 |
| ALPRPIRNLE | ALDH1A3 | 10 | 55 |
| AVFTKNLDK | ALDH1A3 | 9 | 56 |
| VFTKNLDK | ALDH1A3 | 8 | 57 |
| AVFTKNLD | ALDH1A3 | 8 | 58 |
| AAVFTKNLDK | ALDH1A3 | 10 | 59 |
| AVFTKNLDKA | ALDH1A3 | 10 | 60 |

In certain embodiments, the peptide consists of the amino acid sequence shown in one of SEQ ID NOS:1-60. In other embodiments, the peptide is longer, and includes additional amino acid sequence added to one or both ends of the amino acid sequences shown in SEQ ID NOs:1-60. In certain embodiments, the additional amino acid sequence is from the full-length human ALDH1A1 (SEQ ID NO:61) or ALDH1A3 (SEQ ID NO:62) sequence.

The present invention is not limited by the type of cancer stem that is treated in a subject. Examples of cancers include, but are not limited to, lymphomas (e.g., Hodgkin's disease and non-Hodgkin's disease), leukemias (e.g., acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, and chronic lymphocytic leukemia), and sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma). The invention is also applicable to sarcomas and epithelial cancers, such as ovarian cancers and breast cancers.

In certain embodiments, prior to treating a patient with a composition comprising ALDH1 peptide(s) pulsed DC's, a sample from a subject is tested to determine if, (and what type and number) of cancer stem cells the patient possesses. A subject's (e.g., a particular cancer patient's) cancer stem cells (e.g., once isolated and allowed to proliferate in vitro), can be analyzed and screened. For example, in some embodiments, analyzing a subject's cancer stem cells is used as a diagnostic for the subject and as a parameter for the therapeutic efficacy evaluation. Thus, in some embodiments, the present invention provides methods for detection of expression of cancer stem cell biomarkers to identify if the patient has particular cancer stem cells or combinations thereof. In some embodiments, expression is measured directly (e.g., at the nucleic acid or protein level). In some embodiments, expression is detected in tissue samples (e.g., biopsy tissue). In other embodiments, expression is detected in bodily fluids (e.g., including but not limited to, plasma, serum, whole blood, mucus, and urine). In some preferred embodiments, cancer stem cell biomarkers are detected by measuring the levels of the cancer stem cell biomarker in cells and tissue (e.g., cancer cells and tissues). For example, in some embodiments, cancer stem cell biomarkers are monitored using antibodies or by detecting a cancer stem cell biomarker protein/nucleic acid (e.g., CD44, CD24, EpCam, CD49f, ALDH, mir-221, mir-110, and/or mir-93). In some embodiments, detection is performed on cells or tissue after the cells or tissues are removed from the subject. In other embodiments, detection is performed by visualizing the cancer stem cell biomarker in cells and tissues residing within the subject. In some embodiments, cancer stem cell biomarkers are detected by measuring the expression of corresponding mRNA in a tissue sample (e.g., cancerous tissue). In some embodiments, RNA is detected by Northern blot analysis. Northern blot analysis involves the separation of RNA and hybridization of a complementary labeled probe.

In certain embodiments, an additional therapeutic agent is administered with the ALDH1 peptide(s)-DC compositions herein. Any therapeutic agent that can be co-administered with the agents of the present invention, or associated with the agents of the present invention is suitable for use in the methods of the present invention. Some embodiments of the present invention provide methods for administering at least one additional therapeutic agent (e.g., including, but not limited to, chemotherapeutic antineoplastics, antimicrobials, antivirals, antifungals, and anti-inflammatory agents) and/or therapeutic technique (e.g., surgical intervention, radiotherapies). In certain embodiments, therapeutic agent is an immune checkpoint inhibitor, such an a PD-1 inhibitor or PD-L1 inhibitor (e.g., anti-PD-1 and/or anti-PD-L1 mAb). In certain embodiments, the checkpoint inhibitor is atezolizumab, Avelumab, or Durvalumab.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use with the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; 23) radiation; and 24) surgery.

### EXPERIMENTAL

The following example is provided in order to demonstrate and further illustrate certain preferred embodiments and aspects of the present invention and are not to be construed as limiting the scope thereof.

### EXAMPLE 1

### In Vitro ALDH1 Peptide (s)-DC Vaccine Generation

This Examples describes in vitro work conducted to generate dendritic cell—peptide vaccine.

### Material and Methods

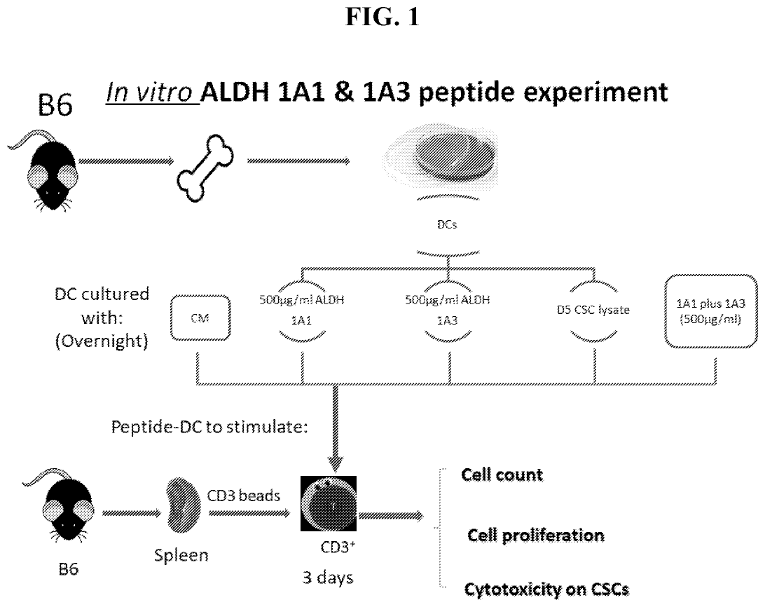

The general procedure for generating ALDH1A1 and/or 1A3 peptide(s) exposed DCs to activate CD3+ T-cells is shown in FIG. 1.

### Preparation of ALDH Peptide(s)-DC

Dendritic cells (DCs) were obtained from bone marrow of normal Female C57BL/6 (B6) mice (Jackson Laboratory). Murine bone marrow-derived cells were cultured in 10-mL complete medium (CM) supplemented with 20 ng/mL GM-CSF, at a concentration of 2-4×10⁵ cells/mL in non-tissue culture petri dishes (Corning). Refresh the half amount CM with GM-CSF on day 3, 6, and 8. On day 10, DCs were loaded with 0.5 mg/ml ALDH 1A1 (SEQ ID NO: 1) or/and 1A3 (SEQ ID NO:6) peptide(s), or ALDH^{high} CSC lysates (as a positive control) and incubated at 37° C. for 24 hours with 5% CO2.

### The Splenetic T Cells Were Primed With ALDH Peptide(s)-DCs

Spleens were harvested from normal B6 mice and were made into splenocytes single suspension. Splenetic T cells were isolated from the splenocytes by MACS separator kits (MiltenyiBiotec. Inc. Auburn, Calif.) including anti-CD3-coupled microbeads. Then splenic CD3+ T cells were co-cultured (activated and expanded) with single or dual ALDH peptide(s)-DCs, or with ALDH^{high} CSC lysate-DCs for 3 days, as shown in FIG. 1.

### CTL Cytotoxicity to ALDH^{high} CSCs Was Examined

We then co-cultured the ALDH^{high} CSCs as target cells with primed splenic T cells as above mentioned for 6 hours. After that, we detected the Cytotoxicity of CTLs by lactate dehydrogenase (LDH) Release Assay (CytoTox 96 Non-Radioactive Cytotoxicity Assay, Promega, Madison, WI) according to the manufacturer's protocol.

### Results

### Cytotoxicity to ALDH^{high} CSCs vs ALDH^{low} non-CSCs of CD3⁺ T Cells Stimulated In Vitro With ALDH 1A1 or/and ALDH 1A3 Peptide (s) DCs

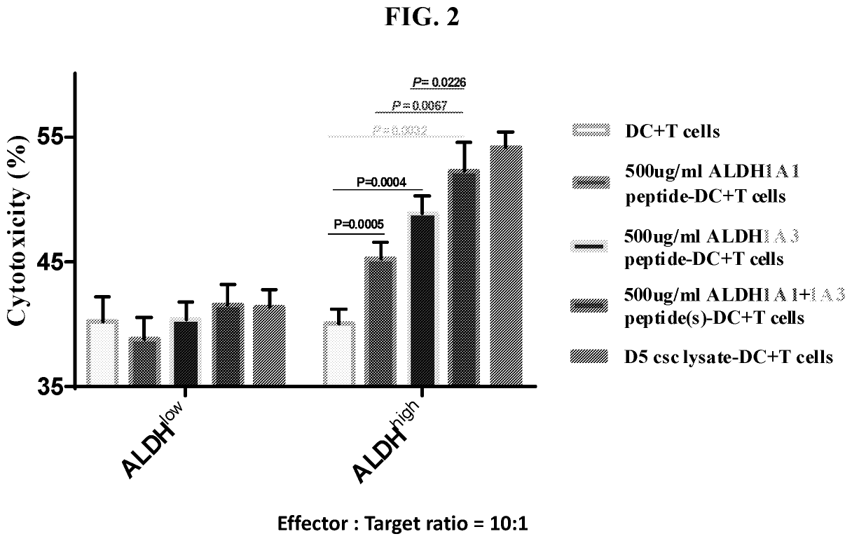

Splenetic CD3⁺ T cells from the normal B6 mice were purified by CD3 Microbeads and were stimulated with PBS, ALDH 1A1 peptide-DC, ALDH1A3 peptide-DC, ALDH 1A1+1A3 peptides-DC, or D5 CSC lysate-DC for 6 hours respectively. Cytotoxicity mediated by such generated CTLs targeting ALDH^{high} CSCs vs ALDH^{low} non-CSCs were measured by LDH release assay. As shown in FIG. 2, CTLs primed with ALDH 1A1 and/or 1A3 peptide(s) exhibits a significant higher killing effect on ALDH^{high} D5 cells than negative control: unloaded-DC primed T cells (all p values<0.05). Importantly, the dual (ALDH 1A1+1A3) peptides-DC-activated T cells significantly kill the ALDH^{high} CSCs higher than single peptide-DC activated T cells (p=0.0067 and p=0.0226 respectively). However, these increased killing effect elicited by ALDH peptide(s) DC-primed T cells were not observed when ALDH^{low} non-CSCs were used as a negative target control.

### EXAMPLE 2

### In Vivo Use of ALDH Peptide(s)-DCs

This Example describes the in vivo use of ALDH peptide (s)-DCs as vaccine in mice.

### Material and Methods

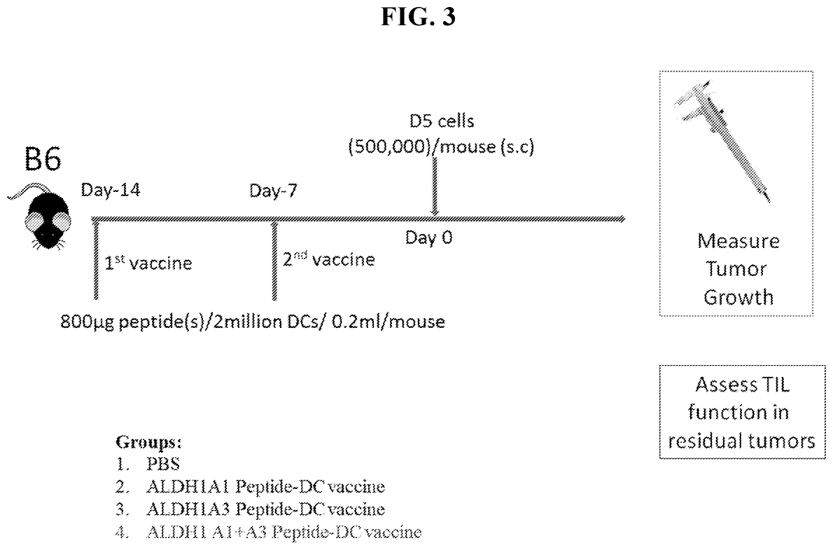

The general protocol for preventing tumor growth in vivo with ALDH peptide (s)-DC vaccine is shown in FIG. 3.

### Establish the ALDH Peptide (s)-DC Vaccine Protective Animal Model

To test the protective effect of ALDH peptide(s)-DC vaccine on melanoma in vivo, corresponding protective animal models were established. All mice were divided into 5 groups and respectively vaccinated twice (on day −14 and day −7) with PBS, ALDH 1A1 peptide-DC, ALDH 1A3 peptide-DC, and ALDH 1A1+1A3 peptides-DC. Each mouse was inoculated subcutaneously with 2×10⁶DCs per vaccine. On day 0, 0.5×10⁶ D5 cells were subcutaneously injected into the flank of each mouse of all as shown in FIG. 3.

### Results

### ALDH 1A1 or 1A3 Peptide-DC Vaccine Demonstrated Significantly Protective Effect on Suppressing D5 Tumor Growth

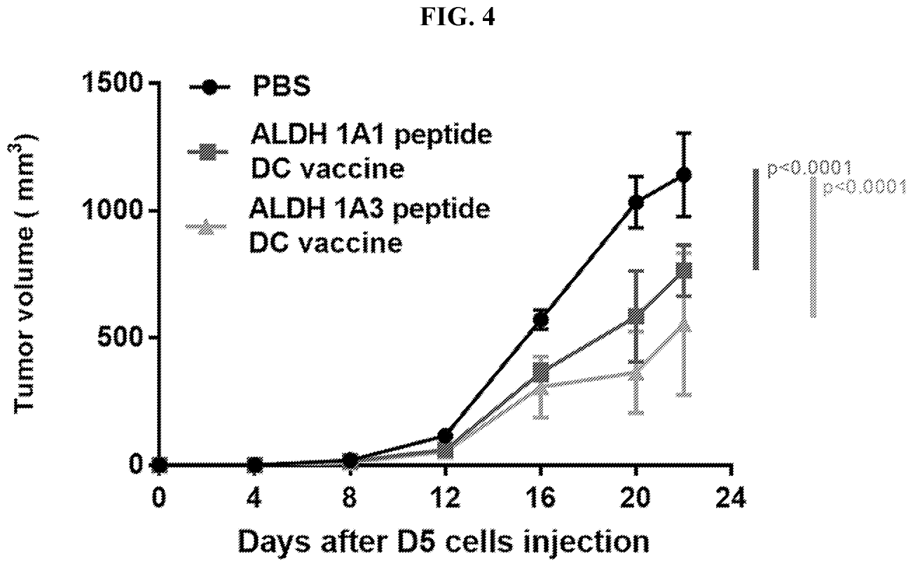

In the ALDH peptide-DC vaccine protective D5 tumor model, two weeks before subcutaneous inoculation of 0.5×10⁶ D5 cells per mouse, mice were vaccinated with different vaccines as indicated in FIG. 3, and the vaccination was repeated after one week. As shown in FIG. 4, ALDH 1A1 or 1A3 peptide-DC vaccine each significantly inhibited subcutaneous tumor growth compared with PBS treated mice (p<0.0001).

### ALDH 1A1 Plus 1A3 Peptides-DC Vaccines Demonstrated Additive Protective Effect on Suppressing D5 Tumor Growth

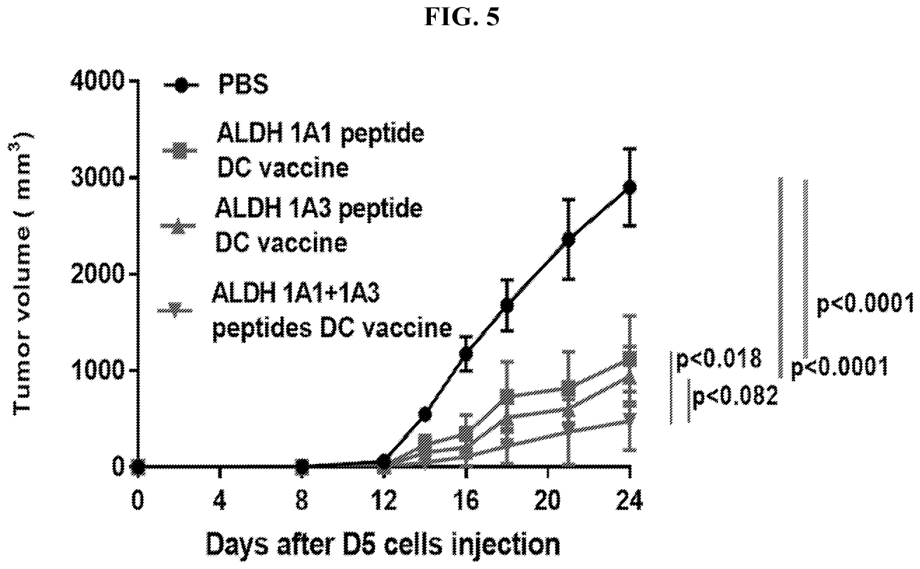

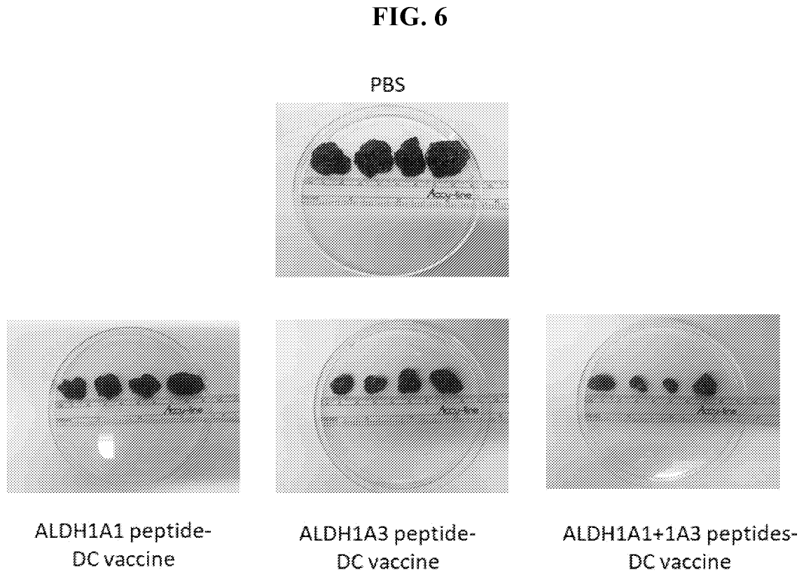

On the basis of above experiment, we tested the effect caused by combined dual ALDH peptides-DC vaccines on tumor growth in protective D5 tumor model. The same as before, twice vaccine were inoculated two weeks before tumor cell injection. As shown in FIG. 5, the ALDH 1A1 or 1A3 peptide-DC vaccine significantly inhibited subcutaneous tumor growth compared with PBS treated mice (p<0.0001), which nicely replicated our early findings as shown in FIG. 4. Importantly, the ALDH 1A1+1A3 peptides-DC vaccine exerted significant (p=0.018) inhibition on the tumor growth compared with single ALDH 1A1 peptide-DC vaccine and markedly more (p=0.082) suppressed the tumor growth when compared with single ALDH 1A3 peptide-DC vaccine. FIG. 6 shows a representative picture of resected tumors at the end of the experiment confirming that the dual peptides-DC vaccine could induce a higher suppression on tumor growth than single peptide-DC vaccine.

### EXAMPLE 3

### Immune Function Assays

This Examples describes immune function assays to correlate the ALDH 1A1 and 1A3 peptide-DC vaccine efficiency.

### Material and Methods

### TILs Expansion and Isolation

The tumors were removed from all mice at the end of the experiments. All the tumors were cut into small piece (1-8 mm³) with further digestion by 1×Collagenase/Hyaluronidase (Stem Cell Technologies) for 30 minutes and finally were made into single cell suspensions. Then the single cells suspensions were cultured in 5 mL complete medium(CM) supplemented with 3000 IU/mL IL-2, at a concentration of 1-2×10⁶cells/mL in non-tissue culture six well (Corning) for 7-10 days. The six well plates were changed with and CM with IL-2 every 3 days. The suspension cells were collected and filtered through 40 μm nylon cell strainers. CD3⁺ TILs were isolated from the suspension cells by MACS separator kits (MiltenyiBiotec. Inc. Auburn, Calif.) as above mentioned.

### Intracellular IFN-γ staining

To determine IFN-γ intracellular secretions, the primed T cells with peptide(s)-DCs as indicted above were permeabilized with pre-chilled Perm Buffer III (BD Bioscience) at 4° C. for 30 min. After washing once with PBS, the cells were stained with FITC-labeled anti-mouse IFN-γ at 4° C. for 30 min. all the samples were monitored using a LSRII flow cytometer (BD Biosciences) and finally analyzed by FlowJo™ version 10 software (Tree Star, Inc., Ashland, Oreg., USA).

### Results

### CD3⁺ TILs From D5 Tumor Bearing Mice Vaccinated With ALDH 1A3 Peptide-DC Demonstrated Significantly Elevated Killing Effect on ALDH^{high} CSCs

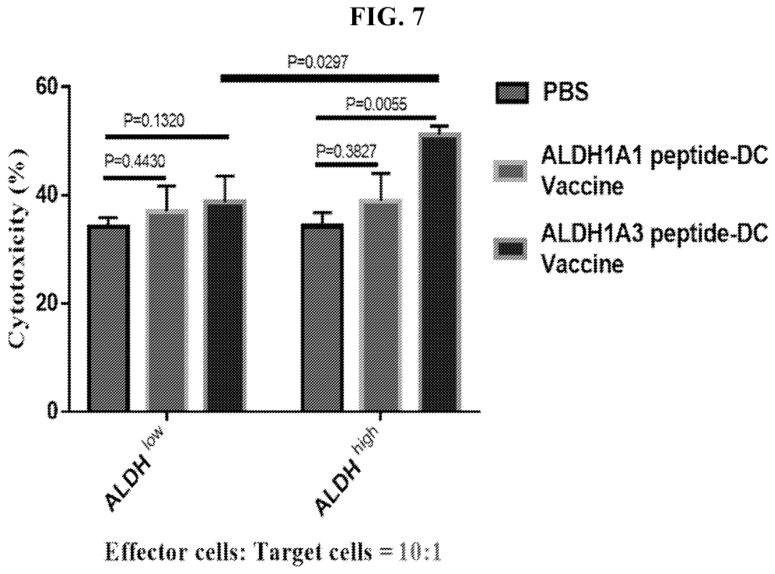

CD3+ TILs were isolated from resected residual tumor tissues from mice vaccinated with PBS, ALDH 1A1 peptide-DC or ALDH 1A3 peptide-DC respectively. After one-week IL-2 expansion, these TILs were incubated with D5 ALDH^{high} CSCs or ALDH^{low} non-CSCs as target cells. Cytotoxicity mediated by CD3⁺ TILs targeting ALDH^{high} CSCs vs ALDH^{low} non-CSCs was measured by LDH release assay. As shown in FIG. 7, CD3⁺TILs from ALDH 1A3 peptide-DC vaccinated mice significantly killed the ALDH^{high} D5 CSCs compared with the PBS control (p=0.0055). Importantly, CD3⁺ TILs from ALDH 1A3 peptide DC-vaccinated mice exhibited a significantly higher killing effect on the ALDH^{high} CSCs than that on ALDH^{low} non-CSCs (p=0.0297).

### ALDH 1A1 +1A3 Peptides-DC Vaccine Confers Splenetic T Cells a Significantly Higher Cytotoxicity to D5 ALDH^{high} CSCs

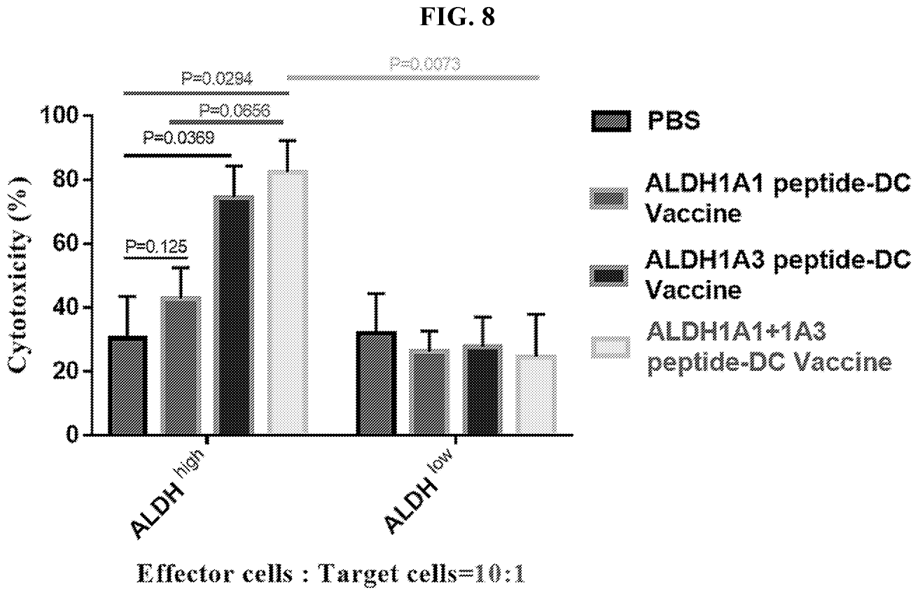

Spleens were harvested from animals subjected to various treatments as indicated (FIG. 8) at the end of the experiments. As shown in FIG. 8, splenetic T cells isolated from ALDH 1A1, 1A3 or 1A1+1A3 peptide(s) DC vaccinated mice exerted stronger killing effects on ALDH^{high} D5 cells respectively (p=0.125, p=0.0369 and p=0.0294) than that splenetic T cells from PBS treated mice at the ratio of E (effect) to T (target) as 10:1. Moreover, the dual (ALDH 1A1+1A3) peptides-DC vaccine displayed a better cytotoxicity to CSCs compared with single peptide (ALDH1A1)-DC vaccine (p=0.0656, nearly p<0.05). Importantly, dual peptides-DC vaccine induces the cytotoxicity to ALDH^{high} CSCs significantly superior to ALDH^{low} non-CSCs (p=0.0073).

### CTL Responses to D5 ALDH^{high} CSCs vs ALDH^{low} Non-CSCs Were Determined By IFN-γ Secretion

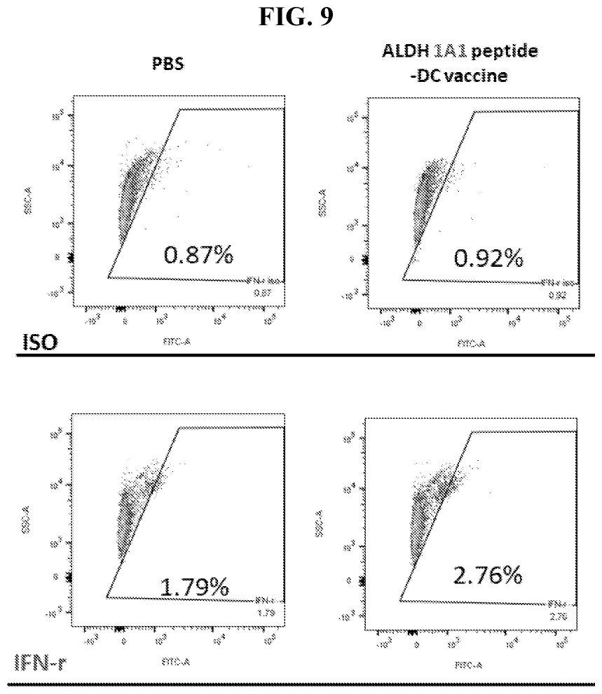

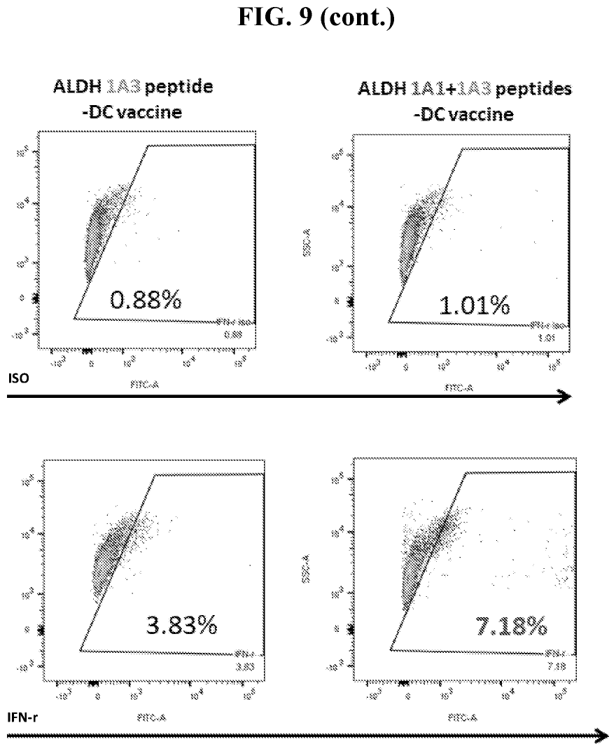

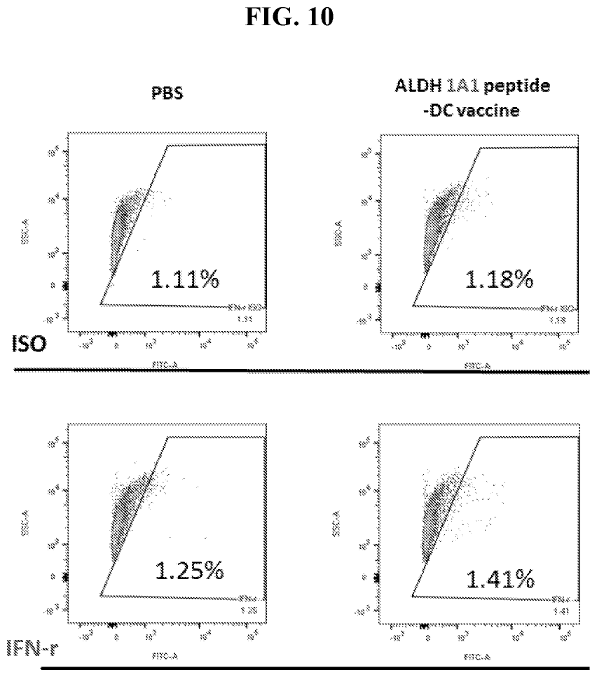

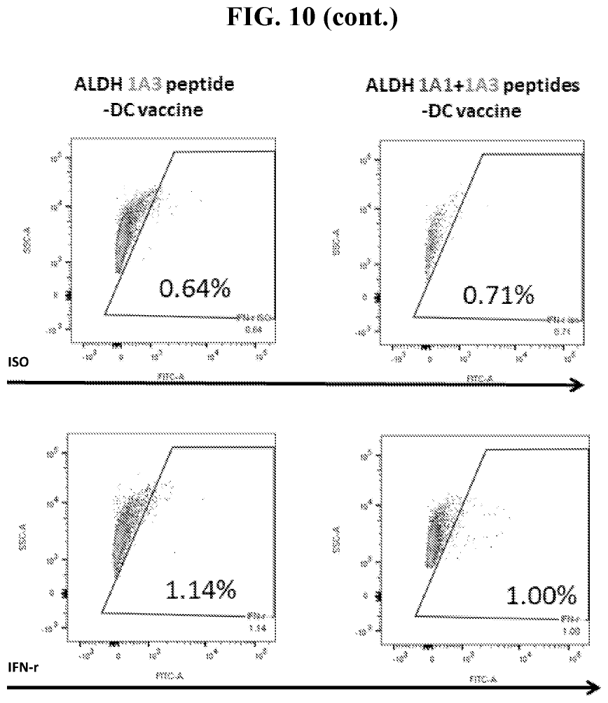

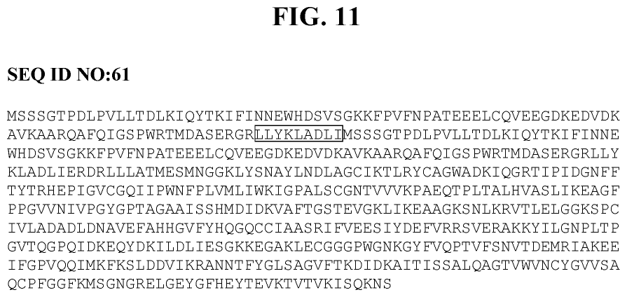

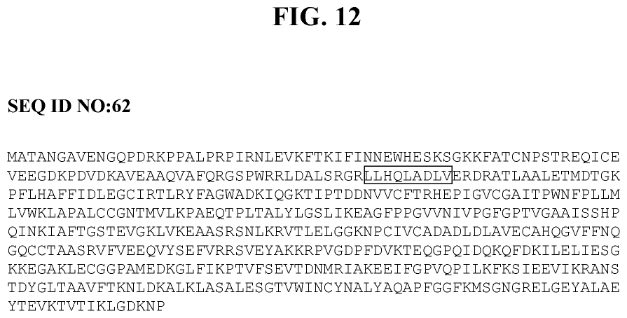

The splenetic CTLs from the different immunized mice were co-cultured with ALDH^{high} CSCs and ALDH^{low} non-CSCs overnight. Then the CTLs were performed intracellular staining with IFN-γ to evaluate the immune response against CSCs vs non-CSCs by flow cytometry analysis. As shown in FIG. 9, compare with the 1.79% IFN-γ intracellular stained T cells from PBS treated mice, an apparently increased proportion of IFN-γ secreting splenic T cells were conferred by ALDH peptide(s):1A1 (2.76%), 1A3(3.83%) and dual 1A1+1A3 (7.18%)-DC vaccines when targeting CSCs. However, these augmented T cell responses cannot be elicited by non-CSCs (FIG. 10).

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A method of treating cancer in a subject comprising:
administering antigen-pulsed dendritic cells (DCs) to a subject having cancer cells such that at least some of said cancer cells are killed,
wherein said antigen-pulsed DCs are initial DCs that have been pulsed in vitro with a composition comprising human ALDH1A1 and/or ALDH1A3 immunogenic peptides that are 8 to 100 amino acids in length,
wherein said composition is free of: i) full-length ALDH1A1 and ALDH1A3 proteins, and ii) tumor cells and cell-lysates.

2. The method of claim 1, wherein said initial DCs comprise immature DCs.

3. The method of claim 1, wherein said human ALDH1A1 and/or ALDH1A3 immunogenic peptides are between 8 and 50 amino acids in length.

4. The method of claim 1, wherein said human ALDH1A1 and/or ALDH1A3 immunogenic peptides are between 8 and 23 amino acids in length.

5. The method of claim 1, wherein said human ALDH1A1 and/or ALDH1A3 immunogenic peptides are between 8 and 10 amino acids in length.

6. The method of claim 1, wherein said composition is further free of ALDH1A1 and ALDH1A3 peptides larger than 100 amino acids in length.

7. The method of claim 1, wherein said composition is further free of ALDH1A1 and ALDH1A3 peptides larger than 35 amino acids in length.

8. The method of claim 1, wherein said composition is further free of ALDH1A1 and ALDH1A3 peptides larger than 10 amino acids in length.

9. The method of claim 1, wherein said ALDH1A1 and/or ALDH1A3 immunogenic peptides comprise or consist of an amino acid sequence shown in SEQ ID NOS:1-60.

10. The method of claim 1, wherein said ALDH1A1 and/or ALDH1A3 immunogenic peptides comprise or consist of the amino acid sequences shown in SEQ ID NOS:1 and/or 6.

11. The method of claim 1, wherein said ALDH1A1 and/or ALDH1A3 immunogenic peptides, collectively, are present in said composition at a concentration of at least 100 μg/ml.

12. The method of claim 1, wherein said initial DCs are from said subject.

13. The method of claim 1, wherein said subject has previously had a solid tumor removed.

14. The method of claim 1, wherein said administering to said subject increases the length of survival of said subject compared to the length of survival without said administering.

15. The method of claim 1, further comprising: administering an immune checkpoint inhibitor to said subject.

16. The method of claim 1, wherein said subject is a human.

17. The method of claim 1, wherein said subject has a cancer selected from the group consisting of: melanoma, breast cancer, prostate cancer, pancreatic cancer, lung cancer, liver cancer, brain cancer, skin cancer, squamous cell carcinoma, and colon cancer.

18. The method of claim 1, further comprising, treating said subject with a chemotherapeutic agent.

19. The method of claim 1, further comprising treating said subject with radiation treatment.

20. The method of claim 1, wherein said cancer cells are cancer stem cells.

21. A method of generating antigen-pulsed dendritic cells comprising:
contacting initial dendritic cells (DCs) in vitro with a composition comprising human ALDH1A1 and/or ALDH1A3 immunogenic peptides that are 8 to 100 amino acids in length,
wherein said composition is free of: i) full-length ALDH1A1 and ALDH1A3 proteins, and ii) tumor cells and cell-lysates.

22. The method of claim 21, wherein said initial DCs comprise immature DCs.

23. The method of claim 21, wherein said human ALDH1A1 and/or ALDH1A3 immunogenic peptides are between 8 and 10 amino acids in length.

24. The method of claim 21, wherein said composition is further free of ALDH1A1 and ALDH1A3 peptides larger than 100 amino acids in length.

25. The method of claim 21, wherein said composition is further free of ALDH1A1 and ALDH1A3 peptides larger than 35 amino acids in length.

26. The method of claim 21, wherein said ALDH1A1 and/or ALDH1A3 immunogenic peptides comprise or consist of an amino acid sequence shown in SEQ ID NOS:1-60.

27. The method of claim 21, wherein said ALDH1A1 and/or ALDH1A3 immunogenic peptides comprise or consist of the amino acid sequences shown in SEQ ID NOS:1 and/or 6.

28. The method of claim 21, wherein said ALDH1A1 and/or ALDH1A3 immunogenic peptides, collectively, are present in said composition at a concentration of at least 100 μg/ml.

29. The method of claim 21, further comprising, prior to said peptide(s) contacting, i) collecting said initial DCs from a subject and, ii) culturing said initial DCs with IL-4 and/or GM-CSF.

30. The method of claim 29, wherein said collecting comprises isolating said initial DCs from blood or bone marrow from said subject.

31. A composition comprising: dendritic cells (DCs), and human ALDH1A1 and/or ALDH1A3 immunogenic peptides that are 8 to 100 amino acids in length,
wherein said composition is free of: i) full-length ALDH1A1 and ALDH1A3 proteins, and ii) tumor cells and cell-lysates.

32. The composition of claim 31, wherein said human ALDH1A1 and/or ALDH1A3 immunogenic peptides are between 8 and 35 amino acids in length.

33. The composition of claim 31, wherein said human ALDH1A1 and/or ALDH1A3 immunogenic peptides are between 8 and 10 amino acids in length.

34. The composition of claim 31, wherein said composition is further free of ALDH1A1 and ALDH1A3 peptides larger than 100 amino acids in length.

35. The composition of claim 31, wherein said composition is further free of ALDH1A1 and ALDH1A3 peptides larger than 35 amino acids in length.

36. The composition of claim 31, wherein said ALDH1A1 and/or ALDH1A3 immunogenic peptides comprise or consist of an amino acid sequence shown in SEQ ID NOS:1-60.

37. The composition of claim 31, wherein said ALDH1A1 and/or ALDH1A3 immunogenic peptides comprise or consist of the amino acid sequences shown in SEQ ID NOS:1 and/or 6.

38. The composition of claim 31, wherein said ALDH1A1 and/or ALDH1A3 immunogenic peptides, collectively, are present in said composition at a concentration of at least 100 μg/ml.

39. A composition comprising: antigen-pulsed DCs which are initial DCs that have been pulsed in vitro with a pulsing composition comprising human ALDH1A1 and/or ALDH1A3 immunogenic peptides that are 8 to 100 amino acids in length, wherein said pulsing composition is free of: i) full-length ALDH1A1 and ALDH1A3 proteins, and ii) tumor cells and cell-lysates.

40. The composition of claim 39, further comprising a physiologically tolerable buffer.

41. The composition of claim 39, wherein said human ALDH1A1 and/or ALDH1A3 immunogenic peptides are between 8 and 35 amino acids in length.

42. The composition of claim 39, wherein said human ALDH1A1 and/or ALDH1A3 immunogenic peptides are between 8 and 10 amino acids in length.

43. The composition of claim 39, wherein said composition is further free of ALDH1A1 and ALDH1A3 peptides larger than 100 amino acids in length.

44. The composition of claim 39, wherein said composition is further free of ALDH1A1 and ALDH1A3 peptides larger than 35 amino acids in length.

45. The composition of claim 39, wherein said ALDH1A1 and/or ALDH1A3 immunogenic peptides comprise or consist of an amino acid sequence shown in SEQ ID NOS:1-60.

46. The composition of claim 39, wherein said ALDH1A1 and/or ALDH1A3 immunogenic peptides comprise or consist of the amino acid sequences shown in SEQ ID NOS:1 and/or 6.

47. The composition of claim 39, wherein said ALDH1A1 and/or ALDH1A3 immunogenic peptides, collectively, are present in said composition at a concentration of at least 100 μg/ml.

48. A system or kit comprising:
a) dendritic cells (DCs), and
b) a composition comprising human ALDH1A1 and/or ALDH1A3 immunogenic peptides that are 8 to 100 amino acids in length, wherein said composition is free of: i) full-length ALDH1A1 and ALDH1A3 proteins, and ii) tumor cells and cell-lysates.

49. The system or kit of claim 48, wherein said composition further comprises a physiologically tolerable buffer.

50. The system or kit of claim 48, wherein said human ALDH1A1 and/or ALDH1A3 immunogenic peptides are between 8 and 35 amino acids in length.

51. The system or kit of claim 48, wherein said human ALDH1A1 and/or ALDH1A3 immunogenic peptides are between 8 and 10 amino acids in length.

52. The system or kit of claim 48, wherein said composition is further free of ALDH1A1 and ALDH1A3 peptides larger than 100 amino acids in length.

53. The system or kit of claim 48, wherein said composition is further free of ALDH1A1 and ALDH1A3 peptides larger than 35 amino acids in length.

54. The system or kit of claim 48, wherein said ALDH1A1 and/or ALDH1A3 immunogenic peptides comprise or consist of an amino acid sequence shown in SEQ ID NOS:1-60.

55. The system or kit of claim 48, wherein said ALDH1A1 and/or ALDH1A3 immunogenic peptides comprise or consist of the amino acid sequences shown in SEQ ID NOS:1 and/or 6.

56. The system or kit of claim 48, wherein said ALDH1A1 and/or ALDH1A3 immunogenic peptides, collectively, are present in said composition at a concentration of at least 100 μg/ml.

57. The system or kit of claim 48, wherein said DCs comprise immature DCs.

58. The system or kit of claim 48, further comprising: c) culture medium comprising IL-4 and/or GM-CSF.


SEQUENCE LISTING (62 sequences)

SEQ ID NO: 1 - protein, 9 aa, Artificial sequence
  Synthetic peptide
LLYKLADLI

SEQ ID NO: 2 - protein, 8 aa, Artificial sequence
  Synthetic peptide
LLYKLADL

SEQ ID NO: 3 - protein, 8 aa, Artificial sequence
  Synthetic peptide
LYKLADLI

SEQ ID NO: 4 - protein, 10 aa, Artificial sequence
  Synthetic peptide
RLLYKLADLI

SEQ ID NO: 5 - protein, 10 aa, Artificial sequence
  Synthetic peptide
LLYKLADLIM

SEQ ID NO: 6 - protein, 9 aa, Artificial sequence
  Synthetic peptide
LLHQLADLV

SEQ ID NO: 7 - protein, 8 aa, Artificial sequence
  Synthetic peptide
LLHQLADL

SEQ ID NO: 8 - protein, 8 aa, Artificial sequence
  Synthetic peptide
LHQLADLV

SEQ ID NO: 9 - protein, 10 aa, Artificial sequence
  Synthetic peptide
RLLHQLADLV

SEQ ID NO: 10 - protein, 10 aa, Artificial sequence
  Synthetic peptide
LLHQLADLVE

SEQ ID NO: 11 - protein, 9 aa, Artificial sequence
  Synthetic peptide
ASERGRLLY

SEQ ID NO: 12 - protein, 8 aa, Artificial sequence
  Synthetic peptide
SERGRLLY

SEQ ID NO: 13 - protein, 8 aa, Artificial sequence
  Synthetic peptide
ASERGRLL

SEQ ID NO: 14 - protein, 10 aa, Artificial sequence
  Synthetic peptide
DASERGRLLY

SEQ ID NO: 15 - protein, 10 aa, Artificial sequence
  Synthetic peptide
ASERGRLLYK

SEQ ID NO: 16 - protein, 9 aa, Artificial sequence
  Synthetic peptide
RLLYKLADL

SEQ ID NO: 17 - protein, 8 aa, Artificial sequence
  Synthetic peptide
LLYKLADL

SEQ ID NO: 18 - protein, 8 aa, Artificial sequence
  Synthetic peptide
RLLYKLAD

SEQ ID NO: 19 - protein, 10 aa, Artificial sequence
  Synthetic peptide
GRLLYKLADL

SEQ ID NO: 20 - protein, 10 aa, Artificial sequence
  Synthetic peptide
RLLYKLADLI

SEQ ID NO: 21 - protein, 9 aa, Artificial sequence
  Synthetic peptide
ASERGRLLY

SEQ ID NO: 22 - protein, 8 aa, Artificial sequence
  Synthetic peptide
SERGRLLY

SEQ ID NO: 23 - protein, 8 aa, Artificial sequence
  Synthetic peptide
ASERGRLL

SEQ ID NO: 24 - protein, 10 aa, Artificial sequence
  Synthetic peptide
DASERGRLLY

SEQ ID NO: 25 - protein, 10 aa, Artificial sequence
  Synthetic peptide
ASERGRLLYK

SEQ ID NO: 26 - protein, 9 aa, Artificial sequence
  Synthetic peptide
KLIKEAAGK

SEQ ID NO: 27 - protein, 8 aa, Artificial sequence
  Synthetic peptide
LIKEAAGK

SEQ ID NO: 28 - protein, 8 aa, Artificial sequence
  Synthetic peptide
KLIKEAAG

SEQ ID NO: 29 - protein, 10 aa, Artificial sequence
  Synthetic peptide
GKLIKEAAGK

SEQ ID NO: 30 - protein, 10 aa, Artificial sequence
  Synthetic peptide
KLIKEAAGKS

SEQ ID NO: 31 - protein, 9 aa, Artificial sequence
  Synthetic peptide
GLSAGVFTK

SEQ ID NO: 32 - protein, 8 aa, Artificial sequence
  Synthetic peptide
LSAGVFTK

SEQ ID NO: 33 - protein, 8 aa, Artificial sequence
  Synthetic peptide
GLSAGVFT

SEQ ID NO: 34 - protein, 10 aa, Artificial sequence
  Synthetic peptide
YGLSAGVFTK

SEQ ID NO: 35 - protein, 10 aa, Artificial sequence
  Synthetic peptide
GLSAGVFTKD

SEQ ID NO: 36 - protein, 9 aa, Artificial sequence
  Synthetic peptide
ALYLGSLIK

SEQ ID NO: 37 - protein, 8 aa, Artificial sequence
  Synthetic peptide
LYLGSLIK

SEQ ID NO: 38 - protein, 8 aa, Artificial sequence
  Synthetic peptide
ALYLGSLI

SEQ ID NO: 39 - protein, 10 aa, Artificial sequence
  Synthetic peptide
TALYLGSLIK

SEQ ID NO: 40 - protein, 10 aa, Artificial sequence
  Synthetic peptide
ALYLGSLIKE

SEQ ID NO: 41 - protein, 9 aa, Artificial sequence
  Synthetic peptide
ALAEYTEVK

SEQ ID NO: 42 - protein, 8 aa, Artificial sequence
  Synthetic peptide
LAEYTEVK

SEQ ID NO: 43 - protein, 8 aa, Artificial sequence
  Synthetic peptide
ALAEYTEV

SEQ ID NO: 44 - protein, 10 aa, Artificial sequence
  Synthetic peptide
YALAEYTEVK

SEQ ID NO: 45 - protein, 10 aa, Artificial sequence
  Synthetic peptide
ALAEYTEVKT

SEQ ID NO: 46 - protein, 9 aa, Artificial sequence
  Synthetic peptide
RLLHQLADL

SEQ ID NO: 47 - protein, 8 aa, Artificial sequence
  Synthetic peptide
LLHQLADL

SEQ ID NO: 48 - protein, 8 aa, Artificial sequence
  Synthetic peptide
RLLHQLAD

SEQ ID NO: 49 - protein, 10 aa, Artificial sequence
  Synthetic peptide
GRLLHQLADL

SEQ ID NO: 50 - protein, 10 aa, Artificial sequence
  Synthetic peptide
RLLHQLADLV

SEQ ID NO: 51 - protein, 9 aa, Artificial sequence
  Synthetic peptide
ALPRPIRNL

SEQ ID NO: 52 - protein, 8 aa, Artificial sequence
  Synthetic peptide
LPRPIRNL

SEQ ID NO: 53 - protein, 8 aa, Artificial sequence
  Synthetic peptide
ALPRPIRN

SEQ ID NO: 54 - protein, 10 aa, Artificial sequence
  Synthetic peptide
PALPRPIRNL

SEQ ID NO: 55 - protein, 10 aa, Artificial sequence
  Synthetic peptide
ALPRPIRNLE

SEQ ID NO: 56 - protein, 9 aa, Artificial sequence
  Synthetic peptide
AVFTKNLDK

SEQ ID NO: 57 - protein, 8 aa, Artificial sequence
  Synthetic peptide
VFTKNLDK

SEQ ID NO: 58 - protein, 8 aa, Artificial sequence
  Synthetic peptide
AVFTKNLD

SEQ ID NO: 59 - protein, 10 aa, Artificial sequence
  Synthetic peptide
AAVFTKNLDK

SEQ ID NO: 60 - protein, 10 aa, Artificial sequence
  Synthetic peptide
AVFTKNLDKA

SEQ ID NO: 61 - protein, 597 aa, Homo sapiens
MSSSGTPDLPVLLTDLKIQYTKIFINNEWHDSVSGKKFPVFNPATEEELCQVEEGDKEDVDKAVKAARQAFQIGSPWRTMDASERGRLLYKLADLIMSSSGTPDLPVLLTDLKIQYTKIFINNEWHDSVSGKKFPVFNPATEEELCQVEEGDKEDVDKAVKAARQAFQIGSPWRTMDASERGRLLYKLADLIERDRLLLATMESMNGGKLYSNAYLNDLAGCIKTLRYCAGWADKIQGRTIPIDGNFFTYTRHEPIGVCGQIIPWNFPLVMLIWKIGPALSCGNTVVVKPAEQTPLTALHVASLIKEAGFPPGVVNIVPGYGPTAGAAISSHMDIDKVAFTGSTEVGKLIKEAAGKSNLKRVTLELGGKSPCIVLADADLDNAVEFAHHGVFYHQGQCCIAASRIFVEESIYDEFVRRSVERAKKYILGNPLTPGVTQGPQIDKEQYDKILDLIESGKKEGAKLECGGGPWGNKGYFVQPTVFSNVTDEMRIAKEEIFGPVQQIMKFKSLDDVIKRANNTFYGLSAGVFTKDIDKAITISSALQAGTVWVNCYGVVSAQCPFGGFKMSGNGRELGEYGFHEYTEVKTVTVKISQKNS

SEQ ID NO: 62 - protein, 512 aa, Homo sapiens [sequence omitted]